(19) United States
(12) Patent Application Publication
(10) Pub. No.: US 2023/0142275 A1
(43) Pub. Date: May 11, 2023

(54) FULL-FACE RESPIRATORY MASK

(71) Applicant: Corporación De Ciencia Y Tecnología Para El Desarrollo De La Industria Naval, Marítima Y Fluvial -CO, Bogota D.C. (CO)

(72) Inventors: Luis Enrique ARANIBAR CAMPO, Cartagena (CO); Priscila AREIZA FRIERI, Cartagena (CO); Manuel Alejandro ARIZA ZULUAGA, Cartagena (CO); Juan David CASTAÑO BUSTOS, Cartagena (CO); Christian Camilo GAMEZ SÁNCHEZ, Bogota D.C. (CO); Julián HERNÁNDEZ CORREDOR, Cartagena (CO); David Leonardo PÉREZ AVELLA, Cartagena (CO); Leonardo Alexander VELASCO PEÑA, Bogota D.C. (CO); Camilo Enrique GÓMEZ CORTÉS, Cartagena (CO)

(21) Appl. No.: 17/802,951

(22) PCT Filed: Jul. 30, 2021
(86) PCT No.: PCT/CO2021/000006

§ 371 Date: Aug. 28, 2022

(30) Foreign Application Priority Data
Jul. 31, 2020 (CO) NC2020/0009612

(51) Int. Cl. A62B 18/02; A62B 18/08; A41D 13/11; A62B 9/04

(52) U.S. Cl. CPC A62B 18/02; A62B 18/084; A41D 13/11; A62B 9/04

(57) ABSTRACT

The present invention corresponds to a full-face respiratory mask that prevents the entry and exit of particles during the breathing process. The mask has a hermetic seal against the skin that does not allow particles found in the environment to pass through and a single opening that is also hermetically sealed, this opening is coupled with the outer filter and is where the filtered air enters and exits.

### FIELD OF THE INVENTION

The present invention is related to full face respiratory masks, which seal the contour of the face to prevent the entry of particles. Particularly, the present invention is related to full face respiratory masks that are coupled to gas supply or particulate filters.

### DESCRIPTION OF THE STATE OF THE ART

Full face respiratory masks have been used in many industries for different purposes. In general, the use of these masks is common to avoid intoxication when handling dangerous substances, firefighters and armed forces also use this type of mask in situations where there may be presence of pollutants in the air. Also, full-face respiratory masks are used in hospitals in the treatment of patients with some type of respiratory problem.

In particular, respiratory masks have been used to assist in the recovery processes of patients with respiratory symptoms of the SARS-CoV-2 virus in the context of the COVID-19 pandemic. Where in these patients, the respiratory masks, in addition to preventing the entry of particles, must also prevent the particles of the SARS-CoV-2 virus from leaving the mask. The above to prevent people in contact with the patient from becoming infected.

Additionally, there is a need for citizens infected with the SARS-CoV-2 virus who are not hospitalized and non-infected citizens to use this type of respiratory mask. In the case of the infected but not hospitalized to prevent the spread of the virus, and in the case of the non-infected citizens to prevent the spread of infection. The above, ensuring that the citizen has adequate breathing.

However, conventional face respiratory masks have sealing drawbacks, among others, because most of these masks have at least one inhalation hole and at least one exhalation hole, and exhalation valves are regularly used in said exhalation holes. The exhalation valves are sealed by negative pressure, which generates a sealing latency time that can allow the entry of particles such as the SARS-CoV-2 virus.

Therefore, there is a need for full face respiratory masks that do not use exhalation valves and have a seal that prevents the entry and exit of particles.

On the other hand, in the state of the art, documents related to full face respiratory masks that prevent the entry of particles that do not use exhalation valves are identified, for example, U.S. Pat. No. 8,955,516B2, US20170334531A1 and U.S. Pat. No. 8,573,217B2.

The document U.S. Pat. No. 8,955,516B2 discloses a respirator including a mask having a body extending between a leading edge and a trailing edge, and a facial seal that extends inward from the body. The face seal has a first wall extending from the body and a second wall intersecting the first wall at a sealing area. The sealing area is configured to contact the user's face to form a continuous circumferential seal. The face seal is folded such that the first and the second wall extend from the sealing area in a common direction. Optionally, the faceseal can be U-shaped.

The respirator of U.S. Pat. No. 8,955,516B2 also includes a lens that is held in the mask, said lens has an attached air purifying cartridge, said purifying cartridge is connected to a gas supply. On the other hand, the mask is adapted to be secured to the wearer's face by a head harness. Additionally, the respirator of U.S. Pat. No. 8,955,516B2 includes a cup that surrounds the user's mouth and nose, said cup forms a breathing channel with the lens that has an attached air-purifying cartridge, where the cup is located behind the lens.

In particular, document U.S. Pat. No. 8,955,516B2 discloses that an exhalation passage with its respective exhalation valve can optionally be used, however, U.S. Pat. No. 8,955,516B2 mentions that an exhalation passage may not be needed while the gas inlet could be designed to also handle the part-time exhalation gas outlet, that is, the gas enters and exits through a single passage.

However, although document U.S. Pat. No. 8,955,516B2 discloses that the gas inlet could be designed to also handle the exit of exhalation gases, document U.S. Pat. No. 8,955,516B2 does not disclose that this gas inlet has any type of sealing. Document U.S. Pat. No. 8,955,516B2 limits itself to indicating that an air purifying cartridge is coupled to the lens but does not mention that some type of hermetic seal is made between the elements to prevent particles from entering through the coupling.

For its part, document US20170334531A1 discloses a diving mask and snorkel structure that includes a full face mask, a frame, at least one lens, a joint, a breathing tube and at least one safety belt. The full face mask includes a first region of soft material connected to a second region of soft material. The frame is combined with the second soft material region of the full face mask. At least one lens is arranged in the frame. The joint is disposed on the full face mask and adjacent to the upper side of the frame. The breathing tube is arranged over the joint and establishes air communication with the full face mask through the joint. At least one seat belt includes a side buckle member for snap-fitting onto the full face mask.

However, document US20170334531A1 discloses that the joint connects a respiratory tube, nevertheless, the document does not mention that this junction between the joint and the respiratory tube has some type of hermetic seal to prevent particles from entering through the junction.

On the other hand, document U.S. Pat. No. 8,573,217B2 discloses a mask assembly for supplying gas to a patient that includes a mask body and a breathing circuit interface. The body of the mask includes an opening for receiving the gas and includes a sealing structure to sealingly engage the patient's face and surround at least the patient's nose and mouth. The breathing circuit interface includes a first portion rotatably connected to the mask body and a second portion that is constructed and arranged to releasably connect to a conduit for delivering gas to the patient through the opening.

In particular, document U.S. Pat. No. 8,573,217B2 discloses that the first portion at one of its ends on its external surface has grooves that extend towards the breathing circuit interface, said grooves provide a path to allow exhaled gas to escape to the outside.

However, the grooves of the first portion disclosed in document U.S. Pat. No. 8,573,217B2 allow the exhaled gas to escape to the outside without any type of filtering, that is, the breathing circuit interface of document U.S. Pat. No. 8,573,217B2 is not completely hermetically sealed.

Thus, the cited documents disclose particulate-preventing full-face respirator masks that do not use exhalation valves. However, these masks do not disclose that the masks have a tight seal in all areas where there is a possibility of particles entering or leaving the mask.

### BRIEF DESCRIPTION OF THE INVENTION

The present invention corresponds to a full-face respiratory mask, which includes a body, an inner seal connected to the body at the perimeter, with a mouth-nose cup and a transparent shield connected to the inner seal at the perimeter. In particular, the mask has an opening that connects the mouth-nose cup of the inner seal with the outside of the mask, through the transparent shield. On the other hand, the clear shield, inner seal, and the body seal the front of the mask and the inner seal seals the perimeter of the face. Similarly, the mouth-nose cup of the inner seal seals the mouth-nose area inside the mask and seals the opening.

In one configuration of the invention, an outer filter or outer gas tube is attached to the mask. Where, the outer filter or the outer gas tube can be connected to the opening through a coupling.

In one configuration of the invention, the inner seal has a first fold that coincides with a front edge of the body; and a second fold that wraps around the opening.

Finally, in a configuration of the invention, the opening is circular in shape and the diameter of the opening is greater than 25 mm.

### BRIEF DESCRIPTION OF THE FIGURES

FIG. 1 shows a configuration of the invention of the full face respiratory mask comprising a body, an internal seal with a mouth-nose cup, a transparent shield with an opening.

FIG. 2 shows a configuration of the invention of the internal seal of this invention, which comprises a mouth-nose cup with a hole, some grooves, a first fold and a second fold. Additionally, a detail of the front end of the mouth-nose cup can be seen where the second fold and the hole in the mouth-nose cup can be seen.

FIG. 3 shows a configuration of the invention of a full-face respirator mask, where the hermetic seals of the front part of the mask between the body, the internal seal and the transparent shield and the opening between a coupling, can be seen.

FIG. 4A shows in one configuration of the invention, the body made up of an upper section and a lower section before being assembled, the body also has some fasteners.

FIG. 4B shows a configuration of the full face respirator mask invention comprising a harness system to support the user's head.

FIG. 5 shows an exploded view of a configuration of the invention of the full face respiratory mask comprising a body, an internal seal with a mouth-nose cup, a transparent shield with an opening, a harness system, a coupling of filter and an external filter.

FIG. 6 shows a configuration of the invention, a full-face respirator mask comprising some types of couplings to connect external filters or external gas tubes with an opening in the mask.

### DETAILED DESCRIPTION OF THE INVENTION

The present invention is aimed at a full face respiratory mask that prevents the entry and exit of particles of up to 0.3 microns (quality N95 by NIOSH) during the breathing process, the mask has a hermetic seal against the skin that does not allow particles that are in the environment or that come out of the interior of the mask pass through and a single opening that is also hermetically sealed by a filter coupling and an external filter. Optionally it can be hermetically sealed through a tube coupling. This opening is filtered through the filter coupling and an external filter, through which the air enters and exits. In the case of the tube coupling, filtering is carried out in the equipment that supplies the gas. Airtight seals prevent particles from escaping into the environment or entering the mask, such as pathogens like the SARS-CoV-2 virus.

Particles must be understood as a small object to which various physical and chemical properties such as volume or mass can be attributed. These vary widely both in size and quantity, from subatomic particles such as the electron, through microscopic particles such as atoms or molecules, within these are considered the particles of the SARS-CoV-2 virus that can reach a size of up to 0.8 microns, up to macroscopic particles such as gunpowder or other granular materials.

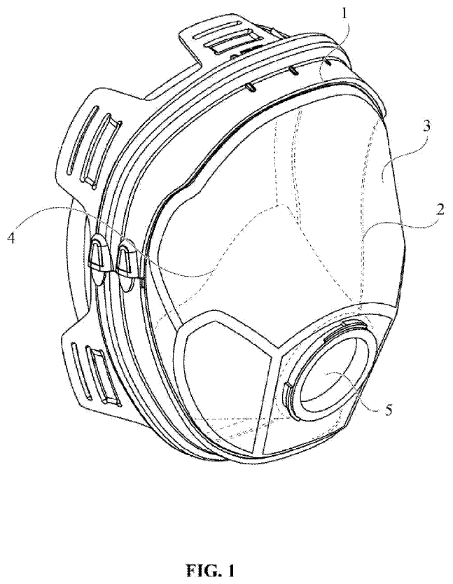

Referring to FIG. 1, in one configuration of the invention, the full face respirator mask includes:
a body (1);
an inner seal (2) perimetraly connected to the body (1), with a mouth-nose cup (4)
a transparent shield (3) perimetraly connected to the inner seal (2).

Where, the mask has an opening (5) that connects the mouth-nose cup (4) of the inner seal (2) with the outside of the mask, through the transparent shield (3).

The full face respirator mask must have a hermetic seal with the environment, this hermetic seal must prevent all particles of size 0.3 microns in diameter that are in the environment from entering the mask. For the above, the full face respirator mask features airtight seals on the parts prone to particle ingress into the mask which are the inside of the mask, the front of the mask, and the opening (5).

It should be understood in the present invention that when we speak of the internal part, we are talking about the part that is towards the user's face and the front part is the part that is facing the outside, in the same way, it is defined as the bottom part, the one that is towards the wearer's chin and the top is the part that is toward the wearer's forehead.

Therefore, the transparent shield (3), the internal seal (2), and the body (1) seal the front part of the mask. Similarly, the inner seal (2) seals the perimeter of the face and the mouth-nose cup (4) of the inner seal (2) seals the mouth-nose area inside the mask and seals the opening (5).

To achieve the sealing of the front part of the mask, the body (1) that is connected to the internal seal (2) must wield a force on the internal seal (2) so that it is imprisoned with the transparent shield (3), forming thus, the hermetic seal on the front part of the mask to prevent the entry of particles from the environment and the exit of particles from the interior of the mask to the environment.

The body (1) is arranged on the internal seal (2) in its perimeter, with which the body (1) can be adjusted to pressure to the internal seal (2), in this way, the needed pressure is generated so that the hermetic seal is formed between the inner seal (2) and the transparent shield (3) to prevent the entry and exit of particles through the front part of the mask.

On the other hand, in one configuration of the invention, the body (1) can be connected to a pressure mechanism that is responsible for imprisoning the body (1) to the inner seal (2), so that it in turn is imprisoned by the transparent shield (3).

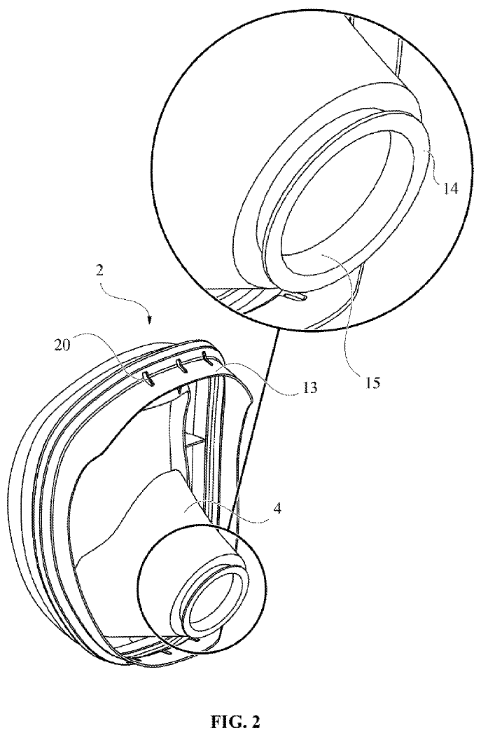

Moreover, the body (1) can be used to align the transparent shield (3) and the internal seal (2) so that they are correctly attached to the mask. Referring to FIGS. 2, 3 and 5, the body (1) can have at least one slot (21) that is configured to receive at least one pin (19) projecting from the transparent shield (3). In this case, as the internal seal (2) is located between the body (1) and the transparent shield (3), said internal seal (2) must also have at least one groove (20). Said at least one groove (20) of the internal seal (2) must coincide with the at least one slot (21) of the body (1) so that the at least one pin (19) passes through at least one slot (20) of the internal seal (2) and are arranged in at least one slot (21) of the body (1). The above allows the body (1), the internal seal (2) and the transparent shield (3) to align, in addition, it allows an easy and quick assembly of the mask.

In one configuration of the invention, the body (1) is made up of at least two sections connected to each other by temporary joints, thereby facilitating the assembly of the mask. In a particular example, the body (1) is made up of an upper section (1A) and a lower section (1B), where the upper section (1A) is connected to the lower section (1B), through a temporary union. In this case, the upper section (1A) and the lower section (1B) are located on the perimeter of the inner seal (2) and are connected through the temporary joint. Optionally, the upper and lower sections (1A, 1B) when connected through the temporary union, imprison the inner seal (2) with the transparent shield (3), which generates the hermetic seal of the front part of the mask. On the other hand, the temporary joint allows the adjustment of the pressure exerted by the upper and lower sections (1A, 1B) to the internal seal (2), for example, if the temporary joint is adjustable straps.

Temporary unions should be understood as those that join together different pieces jointly and form a single piece with them; but that allow, at all times, the separation of the joined pieces, through an easy maneuver that does not damage the elements.

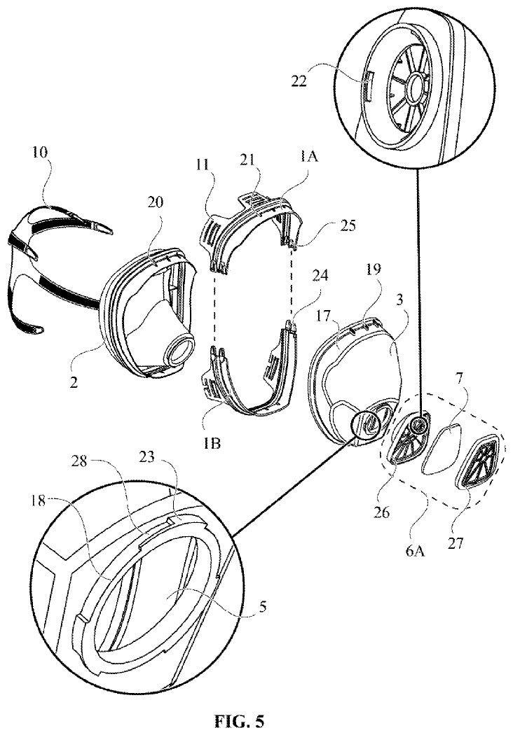

Referring to FIG. 5, when the body (1) is made up of a lower section (1A) and an upper section (1B), an alignment between the upper and lower sections (1A, 1B), the inner seal (2) and the transparent shield (3). For the above, the upper section (1A) can have at least one upper slot (21) and the lower section (1B) can have at least one lower slot (21). Where, when the upper section (1A) and the lower section (1B) face each other, the at least one upper slot (21) and the at least one lower slot (21) are aligned. Therefore, in this mode, the transparent shield (3) has at least two pins (19), where the at least one upper slot (21) is configured to receive one of the at least two pins (19) of the shield. transparent (3) and the at least one lower slot (21) is configured to receive another of the at least two pins (19) of the transparent shield (3).

In this case, as the inner seal (2) is located between the upper and lower sections (1A, 1B) and the transparent shield (3). Said internal seal (2) must also have at least two grooves (20); one of the two grooves (20) must match at least one lower slot (21) and the other of at least two grooves (20) of the internal seal (2) must coincide with at least one upper groove (21). The above, so that at least two pins (19) go through at least two grooves (20) of the internal seal (2) and get arranged in at least one upper slot (21) and at least one bottom slot (21), respectively. As mentioned above, the alignment of the body (1) made up of the upper and lower sections (1A, 1B), the internal seal (2) and the transparent shield (3) allow an easy and quick assembly of the mask.

Temporary joining means can be selected by bayonet type connection, pressure or clip assembly, threaded joints, fasteners (e.g. screws, bolts, nuts, rivets, studs, pins, wedges, clamps, among others), adjustable straps, other types of equivalent temporary unions known to a person of ordinary skill in the art or combinations of the above.

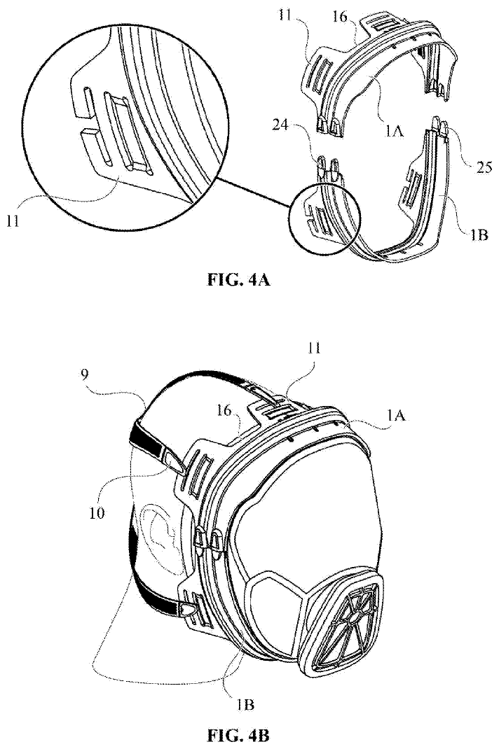

Referring to FIGS. 4A and 5, the temporary joining means is a pressure assembly that is formed with at least one flange (24) disposed on one of the upper or lower sections (1A, 1B), which is configured to fit in at least one housing (25) that is located in the upper or lower complementary sections (1A, 1B). The previous temporary union has the technical effect of facilitating the assembly of the mask, since it is not necessary to use tools to make the temporary union between the upper and lower sections (1A, 1B).

The material of the body (1) can be selected from the group consisting of polypropylene (PP), polyvinyl chloride (PVC); chlorinated polyvinyl chloride (CPVC); polyethylene terephthalate (PET), polyamides (PA) (e.g. PA12, PA6, PA66); polychlorotrifluoroethylene (PCTFE); polyvinylidene fluoride (PVDF); O-ethylene poly tetrafluoride (PTFE); ethylene-chlorotrifluoroethylene (ECTFE); plastics (polyester, vinyl ester, epoxy, vinyl resins) reinforced with fibers (e.g. glass, aramid, polyester), equivalent materials that are known to a person of ordinary skill in the art or combinations of the above. Preferably, the body material (1) is polypropylene (PP) since this material, due to its mechanical and thermal properties, is suitable for injection molding, since it is capable of melting and flowing, in a reversible physical transformation, when subjected to temperatures (melting temperature between 210 and 343° C.) and high pressures (injection pressure between 5.52 and 152 MPa), and taking a specific shape when stabilized (modulus of elasticity between 1.8 and 6 GPa). The stiffness qualities of the material guarantee that the assembly can be adjusted without cracking or breaking (hardness between 50 and 123 Rockwell-M, and 72 and 124 Rockwell-R), with sufficient structure to hold the inner seal together (2) and the transparent shield (3) of the mask, and a degree of deformation (deflection temperature at 0.46 MPa between 40 to 160° C., and at 1.8 MPa between 37 and 172° C.) that allows it to be adjusted in the assembly of the inner seal (2) and the transparent shield (3) without cracking or breaking.

On the other hand, the inner seal (2) is made up of two elements, a frame and a mouth-nose cup (4) that extends towards the front part of the mask. The inner seal frame (2) is what allows a hermetic seal to be formed with the perimeter of the face on the inner part of the mask, the inner seal frame (2) is ergonomically designed in such a way that this fit with the user's face, thereby ensuring a hermetic seal. Referring to FIG. 2, the inner seal frame (2) has a first fold (13) that extends towards the front part of the mask, said first fold (13) is connected with the body (1) on its internal surface.

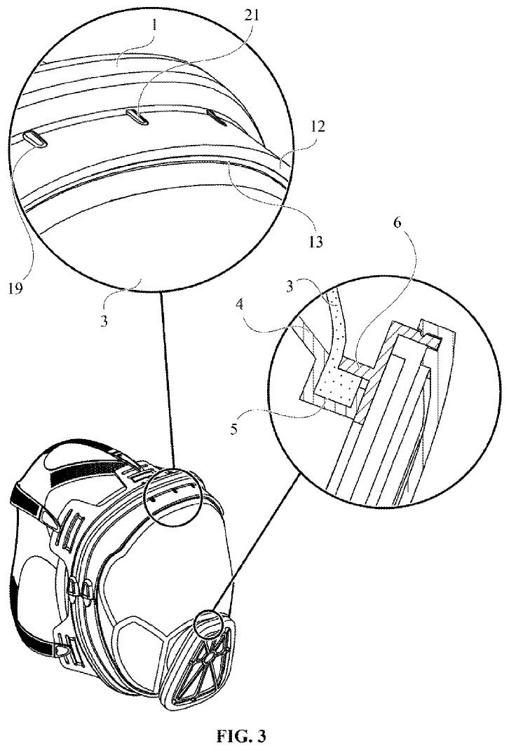

Referring to FIGS. 2 and 3, in one configuration of the invention, the frame of the inner seal (2) has a first fold (13) in its front part. This first fold (13) connects to the body (1) in such a way that the first fold (13) coincides with the front edge (12) of the body (1).

On the other hand, the frame of the inner seal (2) can have a slot on its entire perimeter surface, in said slot the body (1) can be placed. In this mode, the inner seal frame slot (2) covers the front edge (12).

The internal seal frame (2) and the mouth-nose cup (4) form a monolithic body, which means that no connection points are generated where additional sealing must be guaranteed and that the integrity of the seal is not vulnerable to physical wear or mechanical stress due to assembly and disassembly. In addition, by having a monolithic piece, without cracks, referring to FIG. 1, the hermeticity between the transparent shield (3) and the mouth-nasal cup (4) can be guaranteed, thus minimizing the fogging of the transparent shield (3). Another technical effect that the internal seal frame (2) and the mouth-nose cup (4) form a monolithic body is that the number of parts is reduced. Additionally, the fact that the internal seal frame (2) and the mouth-nasal cup (4) are a monolithic piece allows the internal seal (2) to be easily placed on the transparent shield (3) while it molds to the face of the user, with which a double seal is obtained. A first perimeter seal around the face and another over the mouth-nasal cavity without requiring additional structural support elements to achieve it.

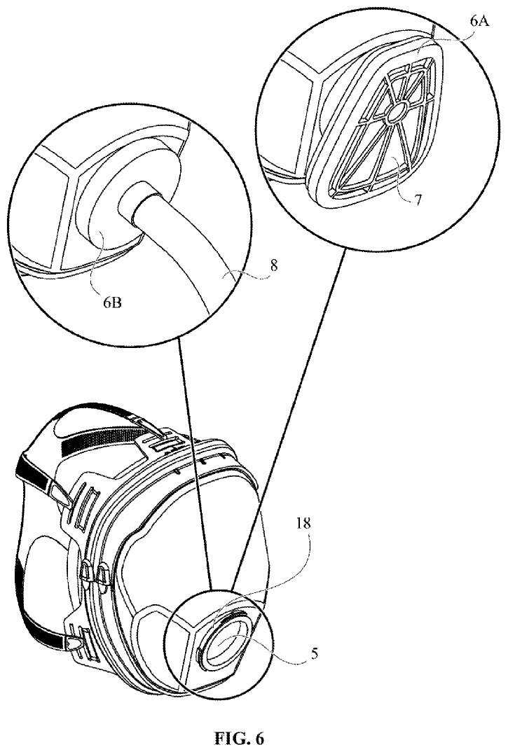

Referring to FIGS. 2 and 6, in one configuration of the invention, the frame of the internal seal (2) has a first fold (13) and, in addition, has the mouth-nasal cup (4) that extends from the frame of the internal seal (2). The inner part of the inner seal frame (2) is ergonomically shaped to fit the wearer's face. In the same way, the mouth-nasal cup (4) in its internal part has an ergonomic shape configured to adjust to the mouth-nasal cavity of the user.

One of the technical effects of sealing the mouth-nasal cavity of the user with the mouth-nasal cup (4) is that it allows inhaled and exhaled air to make a quick transit from the environment and towards the user (inhalation), and from the user to the environment (exhalation), reducing the concentrations of carbon dioxide inside the mask, in the volume generated between the transparent shield (3) and the upper part of the mouth-nasal cup (4). In addition to the above, by reducing the warm air exhaled within the volume that is generated between the transparent shield (3) and the upper part of the mouth-nasal cup (4), the fogging effect that it may have on the transparent shield (3) due to a difference in temperature and humidity between the areas is reduced.

Referring to FIG. 2, the mouth-nose cup (4) of the inner seal (2) is connected to the outside through the opening (5), therefore, the mouth-nose cup (4) has a hole (15) configured to match the opening (5). The hole (15) allows the entry of gas into the mouth-nasal cup (4) so that the user can carry out the breathing process.

The mouth-nasal cup (4) is inserted into the opening (5), through its front end, and thus generates the hermetic seal. The technical effect of generating the hermetic seal in the opening (5) is that it ensures that particles do not enter or exit through this part of the mask. Optionally, an element that can be a coupling imprisons the front end of the mouth-nasal cup (4) against the opening (5) in order to generate the hermetic seal.

Referring to FIG. 2, the mouth-nasal cup (4) can have a second fold (14) at its front end that extends towards the front part of the mask. The second fold (14) is inserted into the opening (5). Said second fold (14) is configured to generate the hermetic seal of the opening (5).

The material of the internal seal (2) is a flexible material that is selected from the group consisting of silicones, natural rubbers, synthetic rubbers, polyurethane, neoprene, thermoplastic elastomers (e.g. thermoplastic olefins (TPE-O or TPO)), Styrenic block copolymers (TPE-S or TPS), vulcanized PP/EPDM compound (TPE-V or TPV), copolyester compound (TPE-E or TPC), thermoplastic polyurethane (TPE-U or TPU), thermoplastic polyamide (TPE-A or TPA), Glossy Thermoplastic Rubber Current—TR, among others), other flexible materials known to a person moderately versed in the matter and combinations of the above. In a particular example, the material of the internal seal (2) is a plastic elastomer (Glossy Thermoplastic Rubber Current—TR), which have rubber-like properties that only differ in temperature resistance, chemical resistance, flexibility (flexural modulus between 0.0150 and 1.18 GPa) and recovery after being subjected to a load (compressive modulus between 0.00196 and 0.0350 GPa). The fundamental characteristics of the families of plastic elastomers are often characterized by their hardness (between 40+/−2 Shore A), cut, scratch, recyclability (in most cases), deformation (elongation between 10.0-74.0%), and thermal resistance (Deflection temperature at 0.46 MPa between 156 and 224° C., and at 1.8 MPa 50 between 210° C.), to abrasion (Abrasion between 0.140-56.0 Taber, mg/1000 cycles) and to wear. Compared to traditional elastomer processes, the saving in time and operations to mold them is substantial, since, with plastic elastomers, it is enough to feed an injection, blow molding or extrusion machine to have a finished piece. In addition, the plastic elastomers are biocompatible and flexible, allowing them to adapt to the shapes of the face to produce the expected seal, while allowing a minimum of comfort for the user without limiting the ability to gesture and communicate without breaking the seal to the environment.

The mask of the present invention comprises the transparent shield (3) that connects with the inner seal (2), this transparent shield (3) allows the user to observe their surroundings. In one configuration of the invention, the transparent shield (3) surrounds the user's face, thereby obtaining a field of vision of at least 90%, which allows the user to have a panoramic view of their surroundings.

Referring to FIG. 5, the transparent shield (3) can have an internal fold (17) and a front fold (18), where the internal fold (17) is connected peripherally to the internal seal (2) to form the hermetic seal of the front part of the mask.

The transparent shield material (3) must allow light to pass through it. The material of the transparent shield (3) can be selected from the group consisting of vitreous material, polycarbonate, polyethylene terephthalate (PET), extrusion polyethylene terephthalate glycol copolyester, polymethyl methacrylate (PMMA), other transparent materials known to a person moderately versed in the matter and combinations of the above. Preferably, the transparent shield material (3) is made of polycarbonate, which is a resistant, transparent material that protects the user from the environment, and has the needed rigidity to achieve the seal that is sought between it, the inner seal (2) and the body (1). Additionally, polycarbonate (PC) is considered the engineering thermoplastic par excellence, due to its combination of toughness (50 to 123 in Rockwell-M and between 72 and 124 in Rockwell-R), high impact resistance (up to −40° C.), high heat deflection temperatures (at 0.46 MPa between 57.2° and 208° C., and at 1.8 MPa between 77.8±187° C.) and transparency, it is similar to glass, colorless and amorphous. Its creep deformation and its chemical resistance is low, as well as its fatigue and wear properties.

In one configuration of the invention, the transparent shield (3) and the body (1) are made of a material that is more rigid than the material of the internal seal (2). The foregoing is to form the hermetic seal on the front part of the mask between two rigid bodies such as the body (1) and the transparent shield (3) and a flexible element that in this case is the internal seal (2), where one of the rigid bodies in this case the body (1) imprisons the flexible element that corresponds to the internal seal (2) against the other rigid body that is the transparent shield (3)

On the other hand, and as mentioned above, the full face respiratory mask comprises an opening (5), which is configured to allow the entry of environment air or a gas that is being supplied to the user and is also configured to allow gas to escape. Optionally, the opening (5) is located in the transparent shield (3) in the lower part of it in the vicinity of the user's mouth. In the modality, where the opening (5) is located in the transparent shield (3), said transparent shield (3) can have a front fold (18) that protrudes from the opening (5) that serves to make the hermetic seal of the opening (5).

In one configuration of the invention, the body (1) has a front section that extends towards the front of the mask, this front section covers the user's mouth and nose area. In this particular case, the opening (5) is located in the front section of the body (1).

The opening (5) has a shape that can be selected from the group made up of polygons (e.g. triangle, quadrilateral, pentagon, hexagon, heptagon, octagon, nonagon, decagon, hendecagon, dodecagon, among others) and closed curves (e.g. circles, ellipses, among others). In a particular example, the opening (5) has a circular shape, said shape facilitates the manufacturing of the opening (5).

To achieve sufficient entry of gas into the interior of the mask, specifically into the interior of the mouth-nasal cup (4), the opening (5) regardless of its shape must have sufficient area to achieve a sufficient flow rate so that the gas enters and exits the mask, ensuring that the user does not have any difficulties in the breathing process. In a particular example, the opening (5) has a circular shape and has a diameter that is greater than 25 mm, which allows a sufficient flow for the gas to enter and exit the mask, and in addition, it decreases the resistance of air inlet and outlet to and from the mask, when it has an external filter attached. Preferably, the opening diameter (5) is in a range from 25 mm to 90 mm, more preferably in a range from 40 mm to 70 mm and even more preferably in a range between 50 mm to 60 mm. In particular, the diameter of the opening (5) is 51.9 mm.

On the other hand, the opening (5) is configured to engage with an element that prevents particles suspended in the environment from entering the masks (e.g. filter) or an element that supplies a medical gas. In one configuration of the invention, the opening (5) is coupled to an external filter (7), in this case, the full face respirator is used by a citizen or by medical staff in an environment where there may be harmful particles due to, for example, the SARS-CoV-2 virus. Additionally, the full face respirator mask has the ability to block particles in both ways, that is, protection is obtained from the user towards the environment and from the environment towards the user. In another configuration of the invention, the opening (5) is coupled to an external gas tube (8), in this case, the user can be, for example, a patient who is receiving some type of medical treatment through his particular use of coupling types to connect external gas tubes, which in turn will be connected to a non-invasive mechanical ventilation system, in this modality, the filtering function is performed by external filters coupled to the non-invasive mechanical ventilator.

The external filter (7) must have the capacity to filter at least 95% of the particles that measure 0.3 microns. Optionally, the external filter (7) can be resistant to oils.

Referring to FIG. 3, the full-face respirator mask may further comprise a coupling (6) connected to the opening (5), where the coupling (6) facilitates the connection, for example, of the external filter (7) or the external medical gas tube (8). In addition, the coupling (6) can form a hermetic seal with the mouth-nose cup (4) of the inner seal (2) and with the transparent shield (3) in the opening (5). Where the mouth-nose cup (4) of the inner seal (2) is inserted into the opening (5) found in the transparent shield (3), the mouth-nose cup (4) has the hole (15) and in said hole (15) is arranged the coupling (6), which generates a pressure, which is responsible for forming the hermetic seal in the opening (5).

Continuing with FIG. 3, in a configuration of the invention, the mouth-nasal cup (4) of the inner seal (2) has the second fold (14), specifically, the second fold (14) can wrap the opening (5) that is located on the transparent shield (3). In FIG. 3, it is observed how the coupling (6) imprisons the second fold (14), which surrounds the opening (5), against the transparent shield (3), thus forming the hermetic seal in the opening (5).

For different applications the coupling (6) can vary its shape depending on the use. For example, when the full-face respirator mask is used by a citizen or by medical staff in an environment where there may be harmful particles, for example the SARS-CoV-2 virus, there must be an element that prevents the entry and exit of particles. Therefore, as mentioned before, in these applications the use of filters is very common, where the coupling (6) can facilitate the connection of said filter.

Referring to FIGS. 5 and 6, in a configuration of the invention, the full face respirator mask to facilitate the connection of the external filter (7) to the opening (5), said external filter (7) is attached by means of a coupling (6) called a filter coupling (6A), which is nothing more than a filter holder. The filter coupling (6A) has a container (26) with a lid (27), where the lid has openings to allow environmental air to enter. The container (26) of the filter coupling (6A) is configured to host the external filter (7), where the filter coupling (6A) having a cover (27) allows the periodic change of the external filter (7) if necessary. The lid (27) is connected to the container (26) through a pressure assembly, in a particular example, the container (26) has a protrusion that protrudes from its outer surface and the lid (27) has a flange that protrudes from its inner surface, where the pressure assembly is made between the lid flange (27) and the container protrusion (26).

In the same way, in applications where the full face respirator mask is used in hospital patients for the supply of medical gases, it is necessary that the outer gas tube (8) be connected to the opening (5), where the gas can be medicinal.

Referring to FIG. 6, in the event that the outer gas tube (8) does not coincide in shape or size with the opening (5), the outer gas tube (8) can be engaged through a coupling (6) called a coupling tube (6B). That is, in case the diameter of the opening (5) in a circular shape is greater than the diameter of the external gas tube (8), the tube coupling (6B) can be a reducing coupling that allows the connection between both elements. Therefore, the tube coupling (6B) can have different shapes that can be selected from the group made up of straight couplings, reducing couplings, male thread couplings, female thread couplings, compression fittings, 90° elbow couplings, 45° elbow couplings, threaded nipples, metallic threaded nipples, concentric reduction, bushing flange, other forms of equivalent couplings known to a person reasonably skilled in the art, or combinations thereof.

The different types of couplings (6) can be connected to the opening (5) by different means of connection, whether temporary or permanent. The joining means can be selected from the group consisting of bayonet type connection, pressure or clip assembly, threaded joints, fasteners (e.g. screws, bolts, nuts, rivets, studs, pins, wedges, clamps, among others), welding, other joints whether temporary or permanent equivalents known to a person moderately versed in the matter, or combinations thereof. Optionally, the coupling (6) is connected to the opening (5) by means of a bayonet-type connection, which is a temporary union that allows a rapid assembly of the elements, where a male element that protrudes from a surface such as pins or flanges fit into a female surface which may be cavities or grooves.

Referring to FIG. 5, in one configuration of the invention, the opening (5) that is located in the transparent shield (3) that has the front fold (18), on the external surface of said front fold (18) some protuberances (22) protrude that form with the external surface of the transparent shield (3) some cavities (28) that have a slope towards the internal part of the mask. On the other hand, the filter coupling (6A) has flanges (23) on its internal surface that are configured to enter the cavities (28) to achieve an assembly between the filter coupling (6A) and the opening (5). Once the flanges (23) of the coupling (6A) fully fit into the cavities (28), these pieces are locked, being a type of connection of the so-called bayonet-type connections.

This connection allows the hermetic seal to be formed in the opening (5), in this case, as the flanges (23) enter the cavities (28) with a slope, the coupling (6A) moves towards the internal part of the mask. When the tabs (23) are fully engaged in the cavities (28), the coupling (6A) imprisons the second fold (14) against the transparent shield (3), thus forming the hermetic seal in the opening (5), as shown in the detail shown in FIG. 3.

The bayonet-type connection type illustrated in FIG. 5, can be replicated for all types of couplings (6), such as the tube coupling (6B).

In one configuration of the invention (not illustrated), the opening (5) located in the transparent shield (3) has longitudinal grooves on its external surface that extend from the front end of the front fold (18) towards the inner part of the mask, where at its opposite end it connects with a radial slot that slopes towards the inner part of the mask. The radial slot is configured to accommodate an element that can be pins. The pins protrude from the coupling (6). Once the pins of the coupling (6) fit into the radial slot, these pieces are locked, being a type of connection of the so-called bayonet type. This connection allows the hermetic seal to be formed in the opening (5), in this case, as the pins enter the radial grooves with a slope, the coupling (6) moves towards the internal part of the mask. When the pins are fully engaged in the radial grooves, the coupling (6) imprisons the second fold (14) against the transparent shield (3) thus forming the hermetic seal in the opening (5).

On the other hand, the full face respirator mask must ensure that the hermetic seal achieved between the inner seal (2) and the face is maintained as long as possible.

Due to the above, the full face respiratory mask may have some element that keeps the mask tight on the face.

Optionally, the body (1) has a harness system (9), which is configured to fit the body on the user's head. The harness system (9) can be made up of at least one elastic band and some fastening elements that are arranged on the body (1), where the elastic bands are connected to the fastening systems. On the other hand, and referring to FIGS. 4A and 4B, the harness system (9) can be made up of a head harness (10) and fastening elements (11) that are arranged on the body (1), where the head harness (10) is connected in the clamping system (11).

The harness system (9) can be connected to the full face respirator mask at at least two attachment points. The more attachment points you have, there is a considerable improvement in the attachment and fit of the full face respirator mask. Referring to FIGS. 4A and 4B, the harness system (9) has five attachment points on the body (1) of the full face respirator mask, where the attachment points are arranged on an inner edge (16) of the body (1).

Referring to FIGS. 5 and 6, the upper section (1A), the lower section (1B), the inner seal (2), the transparent shield (3), the filter coupling (6A) and the tube coupling (6B) can be replaced, assembled or disassembled totally or partially from the full-face respirator mask without the use of tools, so as to allow inspection, cleaning and disinfection. Also, the outer filter (7) can be removed or changed by disassembling the filter coupling (6A). Optionally, said external filter (7) can be exchanged for the same filter or for a filter with similar characteristics that allow filtering particles or mist, oily or non-oily.

### EXAMPLES

### Example 1

Referring to FIGS. 5 and 6, a full-face respiratory mask was designed to prevent the transmission and spread of SARS-CoV-2 virus in the COVID-19 pandemic for medical staff in a hospital with infected patients or for citizens, the specific features of such full-face respiratory mask, are as follows:

In this particular example, the body (1) was made up of two parts, the upper section (1A) and the lower section (1B), connected by means of a temporary union that in this case was a pressure assembly. Referring to FIGS. 4A, 4B and 5, the pressure assembly in this case was made up of four flanges located in the lower section (1B) that were elastically deformed until they fit into four grooves located in the upper section (1A). The upper and lower sections (1A, 1B) were made of polypropylene.

Once the upper and lower sections (1A, 1B) have been assembled, they form a body (1) that is 255 mm long and 198 mm wide.

On the other hand, the inner seal (2) with a mouth-nose cup (4), where the mouth-nose cup has a hole (15) at its front end. The inner seal (2) has a first fold (13) that extends from the front end and a second fold (14) that extends from the front end of the mouth-nose cup (4) and surrounds the hole (15). The inner seal (2) was ergonomically designed to fit the contours of the face and was made of thermoplastic elastomeric material.

For its part, the transparent shield (3) has an opening (5). In addition, the transparent shield (3) has an internal fold (17) and a front fold (18) that extends from the opening (5). The transparent shield (3) is configured to surround the user's face, thus obtaining a field of vision greater than 90%, and was made of polycarbonate.

The opening (5) that is located in the lower part of the transparent shield (3) has a circular shape and measures 51.9 mm. With this size, an adequate flow of air enters the mask for the breathing process.

In the assembly of the elements, the body (1) in its front edge (12) is placed on the first fold (13), and in turn the first fold (13) is placed on the inner edge (17) of the transparent shield. (3). When the upper and lower sections (1A, 1B) are adjusted to form the body (1), a pressure is generated that imprisons the first fold (13) with the internal edge (17) of the transparent shield (3) forming a hermetic seal. On the other hand, the second fold (14) is inserted into the opening (5), in such a way that it coincides with the front fold (18) protruding from the opening (5). In this case an outer filter (7) was press-fitted to the front fold (18) of the transparent shield (3) and to the second fold (14) of the inner seal (2) to form a hermetic seal.

With the full face respirator mask, it was possible to obtain a mask that provides respiratory protection by creating a hermetic seal with the facial perimeter and with the opening that does not allow airborne particles to pass, including pathogens such as viruses and bacteria, only when it is coupled to the external filter. The mask has a designation N95 that is attributed to the external filter (7) attached to the mask, which indicates that the full face mask with the external filter (7) manages to filter at least 95% of the particles found in air of sizes greater than 0.3 microns in diameter.

### Example 2

The full face respirator mask of example 1 was used, but in this case a filter coupling (6A) was used to engage the outer filter (7) to the opening (5). The filter coupling (6A) is a container with a lid, which houses the external filter (7). The external filter (7) selected is the commercial filter 5N11 from the company 3M.

In this example, the filter coupling (6A) and the opening (5) to get connected, they use a bayonet system to connect to each other, therefore, the filter coupling (6A) has flanges (23) on its internal surface, which are configured to enter cavities (28) to achieve the assembly between the filter coupling (6A) and the opening (5). The cavities (28) are formed between some protuberances (22) that protrude from the front fold (18) and the external surface of the transparent shield (3), said cavities (28) have a slope towards the internal part of the mask. Once the flanges (23) of the filter coupling (6A) fully fit into the cavities (28), these pieces are blocked and the hermetic seal of the opening (5) is formed.

With the full-face respirator mask ok example 2, it was possible to obtain a mask that provides respiratory protection by creating a hermetic seal with the facial perimeter and in the opening (5) that does not allow particles that are in the air to pass, between them, pathogens such as viruses and bacteria, only when it is attached to the external filter. The mask has a designation N95 that is attributed to the outer filter (7) attached to the mask, which indicates that the full face mask attached to the outer filter (7) manages to filter at least 95% of the particles found in air of sizes greater than 0.3 microns in diameter.

### Example 3

The full face respirator mask from Example 1 was used, but in this case the full face respirator mask was modified to be used by non-critical patients who are being supplied with medical oxygen. In this case a tube coupling (6B) was used to engage the outer gas tube (8) to the opening (5), where the outer tube was connected to a medical oxygen supply. The tube coupling (6B) is a reducing coupling, which allows the external gas tube (8), which in this case has a smaller diameter than the diameter of the opening (5), to be connected to it. In this mode, the tube coupling (6B) and the opening (5) use a bayonet system to connect to each other, therefore, the tube coupling (6B) has flanges (23) on its internal surface that are configured to enter some cavities (28) to achieve the assembly between the filter coupling (6A) and the opening (5). The cavities (28) are formed between some protuberances (22) that protrude from the front fold (18) and the external surface of the transparent shield (3), said cavities (28) have a slope towards the internal part of the mask. Once the flanges (23) of the coupling (6A) fully fit into the cavities (28), these pieces are blocked and the hermetic seal of the opening (5) is formed.

With the full-face respiratory mask of example 3 assembled with the tube coupling (6B), it was possible to obtain a mask for non-invasive mechanical ventilation of patients requiring respiratory therapy. In this modality, the filtering function must be guaranteed by the filters arranged in the mechanical ventilator, since the mask complies with the tightness between the mask and the user's facial perimeter, as well as between the different parts that make it up.

### Example 4

The full face respirator mask of example 1 was used, but in this case a filter coupling (6A) was used to attach the outer filter (7) in environments that may contain certain oily and non-oily particles, to the opening (5). The filter coupling (6A) is a container with a lid, which hosts the external filter (7). The external filter (7) selected is the commercial filter 5P71 from the company 3M.

In this example, the filter coupling (6A) and the opening (5) to get connected, they use a bayonet system to connect to each other, therefore, the filter coupling (6A) has flanges (23) on its internal surface, which are configured to enter cavities (28) to achieve the assembly between the filter coupling (6A) and the opening (5). The cavities (28) are formed between some protuberances (22) that protrude from the front fold (18) and the external surface of the transparent shield (3), said cavities (28) have a slope towards the internal part of the mask. Once the flanges (23) of the filter coupling (6A) fully fit into the cavities (28), these pieces are blocked and the hermetic seal of the opening (5) is formed.

With the full-face respirator mask in example 4, it was possible to obtain a mask that provides respiratory protection by creating a hermetic seal with the facial perimeter that does not allow airborne particles to pass, including pathogens such as viruses and bacteria, only when it is coupled with the external filter. The mask has a P95 designation that is attributed to the outer filter (7) attached to the mask, which indicates that the full face mask attached to the outer filter (7) manages to filter at least 95% of the particles found in the air of sizes greater than 0.3 microns in diameter, oily or non-oily.

It is to be understood that the present invention is not limited to the modalities described and illustrated, for as will be evident to a person skilled in the art, there are possible variations and modifications that do not deviate from the spirit of the invention, defined by the following claims.

1. A full face respirator mask, comprising:
a body (1);
an inner seal (2) perimetraly connected to the body (1), with a mouth-nose cup (4); and
a transparent shield (3) perimetraly connected to the inner seal (2);
where, the transparent shield (3), the internal seal (2), and the body (1) seal the front part of the mask;
where, the mask has an opening (5) that connects the mouth-nose cup (4) of the inner seal (2) with the outside of the mask, through the transparent shield (3);
where, the inner seal (2) seals the perimeter of the face; and
where the mouth-nose cup (4) of the inner seal (2), seals the mouth-nose area inside the mask and seals the opening (5).

2. The mask according to claim 1, wherein the inner seal (2) has a first fold (13) that coincides with a front edge (12) of the body (1); and a second fold that wraps around the opening (5).

3. The mask according to claim 1, wherein the body (1) is made up of an upper section (1A) and a lower section (1B); where the upper section (1A) is connected to the section (1B), through a temporary joint.

4. The mask according to claim 3, wherein the temporary joint is a pressure assembly that is formed with a flange (24) in one of the upper or lower sections (1A, 1B), configured to fit in a hosting (25) in the complementary upper or lower sections (1A, 1B).

5. The mask according to claim 1, further comprising a coupling (6) connected to the opening (5).

6. The mask according to claim 1, wherein an outer filter (7) is connected to the opening (5); or as an alternative, wherein an outer tube (8) connects to the aforementioned opening (5).

7. The mask according to claim 5, wherein an outer filter (7) is connected to the opening (5); or as an alternative, wherein an outer tube (8) connects to the aforementioned opening (5).

8. The mask according to claim 5, wherein the coupling (6) is connected to the opening (5) by means of a bayonet-type connection.

9. The mask according to claim 1, wherein the diameter of the opening (5) is greater than 25 mm.

10. The mask according to claim 9, wherein the diameter of the opening (5) is 51.9 mm.

11. The mask according to claim 1, wherein the body (1) has a harness system (9), which is configured to fit the body (1) on the head of the user.

12. The mask according to claim 11, where the harness system (9) consists of a head harness (10) and a fastening system (11) that is arranged on the body (1), where the head harness (10) plugs into the fastening system.

13. The mask according to claim 12, wherein the harness system (9) is arranged at five points on the body (1).

14. The mask according to claim 1, wherein the material of the internal seal (2) is selected from the group consisting of silicons, natural rubbers, synthetic rubbers, polyurethane, neoprene, thermoplastic elastomers (e.g. thermoplastic olefins (TPE-O or TPO)), Styrenic block copolymers (TPE-S or TPS), vulcanized PP/EPDM compound (TPE-V or TPV), copolyester compound (TPE-E or TPC), thermoplastic polyurethane (TPE-U or TPU), thermoplastic polyamide (TPE-A or TPA), Glossy Thermoplastic Rubber Current—TR, among others), other flexible materials known to a person moderately versed in the art and combinations of the above.
